(19) United States
(12) Patent Application Publication
(10) Pub. No.: US 2007/0043439 A1
(43) Pub. Date: Feb. 22, 2007

(54) Method of assembling a graphical image biomechanical supplement

(76) Inventor: Theodore J. Becker, Everett, WA (US)

Correspondence Address: LITMAN LAW OFFICES, LTD, PO BOX 15035, CRYSTAL CITY STATION, ARLINGTON, VA, 22215, US

(21) Appl. No.: 11/205,210

(22) Filed: Aug. 17, 2005

(51) Int. Cl. A61F 2/08

(52) U.S. Cl. 623/13.17

(57) ABSTRACT

The method of assembling a graphical image biomechanical supplement invention involves assembling a plurality of biomechanical images selected from an electronic database onto a single document, wherein the biomechanical images are line art silhouettes representing human body postures or positions and specific body part positions. The method of assembling includes selecting at least two biomechanical images from a plurality of biomechanical images, assigning a supplemental description to each of the selected at least two biomechanical images, and compiling the selected at least two biomechanical images and their assigned supplemental descriptions onto a single document. Each of the plurality of biomechanical images may include a brief text description. The selection of biomechanical images may be from a universal biomechanical image database that includes every selectable biomechanical image, or a work specific biomechanical image grouping compiled from a universal biomechanical image database.

### BACKGROUND OF THE INVENTION

1. Field of the Invention

The present invention relates to a computerized method of assembling biomechanical images onto a single document, wherein each biomechanical image comprises line art silhouettes representing human body postures and positions and specific body part positions. Each biomechanical image is associated with both a brief text description included in the image and a supplemental description regarding work conditions associated with that biomechanical image.

2. Description of the Related Art

Typical administrative summary forms relating to employee's physical capacities are generally limited to written descriptions of the employees of physical capacities. Administrative summary forms include physicians estimate of physical capacities, vocational counselors job analysis worksheets, and physical/occupational therapists functional capacity evaluation (FCE) summaries. The critical element in communicating an employee's physical capacities becomes the writer's ability to communicate the appropriate physical capacity guides. For example, physicians typically describe what patients are not capable of doing on the basis of capacity. Although a physician must consider all three parts of the return to work formula (risk, capacity, and tolerance) from the individual worker's point of view, perhaps the greatest amount of guesswork expected of the physician is in the determination of capacity. Employers often request detailed lists of restrictions based on the ability to lift, push, pull, climb, band, stoop, crawl, kneel, and other similar activities. When presented in a form, it is often difficult for a physician to differentiate what a normal healthy person of similar age, sex, education, and body build would be capable of doing. Physician forms do not have the option for selecting a body posture or position or body part position by graphic image.

As a bridge between health care and the workplace, the functional capacity evaluation assists the rehabilitation team in evaluating the patient's recovery with in a vocational context. Typically a physical therapist, an occupational therapist, or a vocational evaluator administers the FCE test battery, which objectively establishes the worker's capacity to perform functional tasks. Simple forms are used to compare the worker's general functional capacity data to the physical demands of a given job. However, these forms again do not have the option for selecting a body posture or position or body part position by graphic image and thus rely merely upon written description.

Another problem with current reporting standards of employee job function evaluations and descriptions is that physicians and health care workers only have a very general understanding of what the specific job requirements are of their patients. In many such instances, the details of the job that are not considered by the physician turn out to be crucial factors in treatment, therapy, and administration of medical advice. Knowing the object of limitations and work capabilities enables the rehabilitation professional or medical provider to present a stronger argument to the potential employer as to why the client is qualified to work, and therefore should be considered for employment.

Rather than relying on the merits of the injured worker's subjective input in determining return to work, increasingly physicians have recognized the need for objective physical findings. The challenge is to come up with the protocol and a process that has a reasonable expectation for delivering the predictive validity of and still retain those qualities of low-cost and practical implementation.

Thus, a simple to employ, yet comprehensive biomechanical supplement standard for validating, illustrating, and describing job capacities regarding employment solving the aforementioned problems is desired.

### SUMMARY OF THE INVENTION

The present invention is directed towards a computerized method of assembling a plurality of biomechanical images onto a single document, wherein the biomechanical images comprise line art silhouettes representing human body postures or positions and specific body part positions. The method of assembling including the steps of selecting at least two biomechanical images from a plurality of biomechanical images, assigning a supplemental description to each of the selected at least two biomechanical images, and, compiling the selected at least two biomechanical images and their assigned supplemental descriptions onto a single document. Each of the plurality of biomechanical images may include a brief text description.

The step of selecting from the plurality of biomechanical images further includes selecting a biomechanical image from the group consisting of: 1) a universal biomechanical image database that includes every selectable biomechanical image, and, 2) a grouping of work specific biomechanical images grouping. The grouping of work specific biomechanical images further comprises pre-selected groupings of biomechanical images selected from either, all varieties of a single titled body posture or position or body part position, body postures and positions or body part positions associated with a particular type of job, appropriate for the stature of a worker, associated with a particular type of equipment used on a job, associated with a company's policy with a particular type of equipment used on a job, or, associated with seasonal changes affecting a job.

The step of assigning the supplemental description to each of the selected at least two biomechanical images further includes either manually inputting the supplemental description to be associated with the biomechanical image, or, choosing the supplemental description from a predetermined list of supplemental descriptions associated with the biomechanical image. The content of the supplemental description is information of either a frequency of operation description associated with a body posture or position or a body part position, a time period description associated with a body posture or position or a body part position, a load associated with a body posture or position or a body part position, and, a minimum or special requirements description associated with a body posture or position or a body part position.

Additionally, the step of compiling the selected at least two biomechanical images further comprises organizing or grouping the at least two biomechanical images into similar body posture or position or body part position categories on the single document.

The single document of selected and grouped biomechanical images of the present invention may be a printed document, a printed placard, an electronic document, an electronic image of a computer generated or printed document, and a database record. The document may be, e.g., a job analysis or a biomechanical supplement to a job analysis for use by employers, vocational counselors, insurers, physicians, attorneys, etc., for evaluating a potential employee's capacity to perform a job, disability claims, insurance premiums, etc.

Another related embodiment of the present invention includes a computer controlled printing apparatus for printing to a printable medium. The printing apparatus prints under computer control at least two biomechanical images comprised of line art silhouettes representing body postures or positions and specific body part positions and a brief text description of the biomechanical image, organizes the biomechanical images for printing on the printable medium into similar body postures or positions and body part position categories, and printing supplemental description fields associated with each of the printed biomechanical images. The printable medium is selected from the group consisting of paper media, rigid or board-type media, transparent projection media, and any media suitable for printed publication or graphical communication.

An additional related embodiment of the present invention includes a computer readable medium capable of storing a graphical representation, the computer readable medium having at least two biomechanical image representations comprised of line art silhouettes representing body postures or positions and specific body part positions, and supplemental descriptions fields for data to be associated with each of the biomechanical image representations, wherein each of the biomechanical image representations includes a brief text data description, and the biomechanical image representations are organized or grouped in the computer readable medium into similar body posture or position or body part position categories. The computer readable medium may be either an electronic document, an electronic image of a computer generated or a printed document, a database record or any computer readable medium suitable for storing and presenting a graphical image.

These and other features of the present invention will become readily apparent upon further review of the following specification and drawings.

### BRIEF DESCRIPTION OF THE DRAWINGS

Other objects of this invention will appear in the following description and appended claims, reference being made to the accompanying drawings forming a part of the specification where in like reference characters designate corresponding parts in the several views.

FIG. 1A is a biomechanical series of images grouped based on hand repetition/hand grasp, fine hand grasp body positions.

FIG. 1B is a biomechanical series of images grouped based on hand repetition/hand grasp, gross hand grasp body positions.

FIG. 1C is a biomechanical series of images grouped based on hand repetition/hand grasp, dryer hand grasp body positions.

FIG. 1D is a biomechanical series of images grouped based on upper extremity (UE) axis, forearm position close body positions.

FIG. 1E is a biomechanical series of images grouped based on upper extremity (UE) axis, forearm position seated/standing body positions.

FIG. 1F is a biomechanical series of images grouped based on seated body, seated upright body positions.

FIG. 1G is a biomechanical series of images grouped based on seated body, seated lean/front/back body positions.

FIG. 1H is a biomechanical series of images grouped based on seated body, seated lean/twist/side/bend body positions.

FIG. 1I is a biomechanical series of images grouped based on floor seated body, seated straight leg body positions.

FIG. 1J is a biomechanical series of images grouped based on floor seated body, seated circle sit body positions.

FIG. 1K is a biomechanical series of images grouped based on cervical, uni-planar body positions.

FIG. 1L is a biomechanical series of images grouped based on cervical, flex/elevate/rotate body positions.

FIG. 1M is a biomechanical series of images grouped based on cervical, protract/retract body positions.

FIG. 1N is a biomechanical series of images grouped based on kneel/squat/crawl, 4×4 kneeling crab body positions.

FIG. 1O is a biomechanical series of images grouped based on kneel/squat/crawl, kneel stand body positions.

FIG. 1P is a biomechanical series of images grouped based on 10 kneel/squat/crawl, kneel squat body positions.

FIG. 1Q is a biomechanical series of images grouped based on kneel/squat/crawl, straddle squat body positions.

FIG. 1R is a biomechanical series of images grouped based on kneel/squat/crawl, squat body positions.

FIG. 1S is a biomechanical series of images grouped based on stand/bend/stoop/crouch, standing body positions.

FIG. 1T is a biomechanical series of images grouped based on stand/bend/stoop/crouch, bend/stoop/crouch body positions.

FIG. 1U is a biomechanical series of images grouped based on carry body positions.

FIG. 1V is a biomechanical series of images grouped based on push/pull body positions.

FIG. 1W is a biomechanical series of images grouped based on push/pull elephant tape body positions.

FIG. 1X is a biomechanical series of images grouped based on climbing body positions.

FIG. 1Y is a biomechanical series of images grouped based on lying, side lying body positions.

FIG. 1Z is a biomechanical series of images grouped based on lying, supine lying body positions.

FIG. 1AA is a biomechanical series of images grouped based on lying, prone lying body positions.

FIG. 1AB is a biomechanical series of images grouped based on reach, standing reach body positions.

FIG. 1AC is a biomechanical series of images grouped based on reach, seated reach body positions.

FIG. 2A is a biomechanical series of images first grouped by body positions associated with typical office work, and further grouped by hand repetition, hand grasp body positions.

FIG. 2B is a biomechanical series of images first grouped by body positions associated with typical office work, and further grouped by seated body positions.

FIG. 2C is a biomechanical series of images first grouped by body positions associated with typical office work, and further grouped by cervical body positions.

FIG. 2D is a biomechanical series of images first grouped by body positions associated with typical office work, and further grouped by kneel/squat/crawl body positions.

FIG. 2E is a biomechanical series of images first grouped by body positions associated with typical office work, and further grouped by stand/bend/stoop/crouch body positions.

FIG. 2F is a biomechanical series of images first grouped by body positions associated with typical office work, and further grouped by reach body positions.

FIG. 3A is a blank biomechanical supplement document for receiving selected biomechanical images and receiving user input to annotate the selected biomechanical images.

FIG. 3B is a blank biomechanical supplement document for receiving selected biomechanical images and receiving user selection of a supplemental description for each of the selected biomechanical images.

FIG. 4A is a biomechanical supplement document having selected biomechanical images and blank user annotation fields proximate each biomechanical image.

FIG. 4B is a biomechanical supplement document having selected biomechanical images and predetermined supplemental description identifiers for each of the selected biomechanical images.

Similar reference characters denote corresponding features consistently throughout the attached drawings.

### DETAILED DESCRIPTION OF THE PREFERRED EMBODIMENTS

The method of the present invention is directed toward assembling under computer control a number of biomechanical images onto a single document. This single document, called hereinafter a biomechanical supplement, provides a summary of graphical images representing body postures or positions and body part positions associated with a particular job function or work environment. The biomechanical supplemental is intended to be an attachment to a job analysis used by employers, vocational counselors, insurers, physicians, attorneys, occupational therapist, physical therapist, and users of function capacity evaluations for evaluating a potential employee's capacity to perform a job, disability claims, insurance premiums, etc.

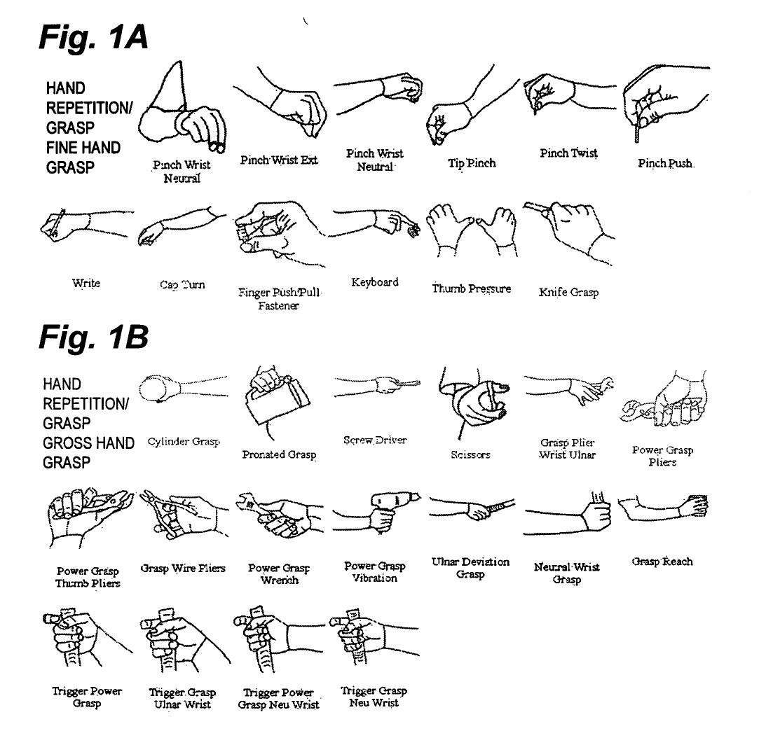

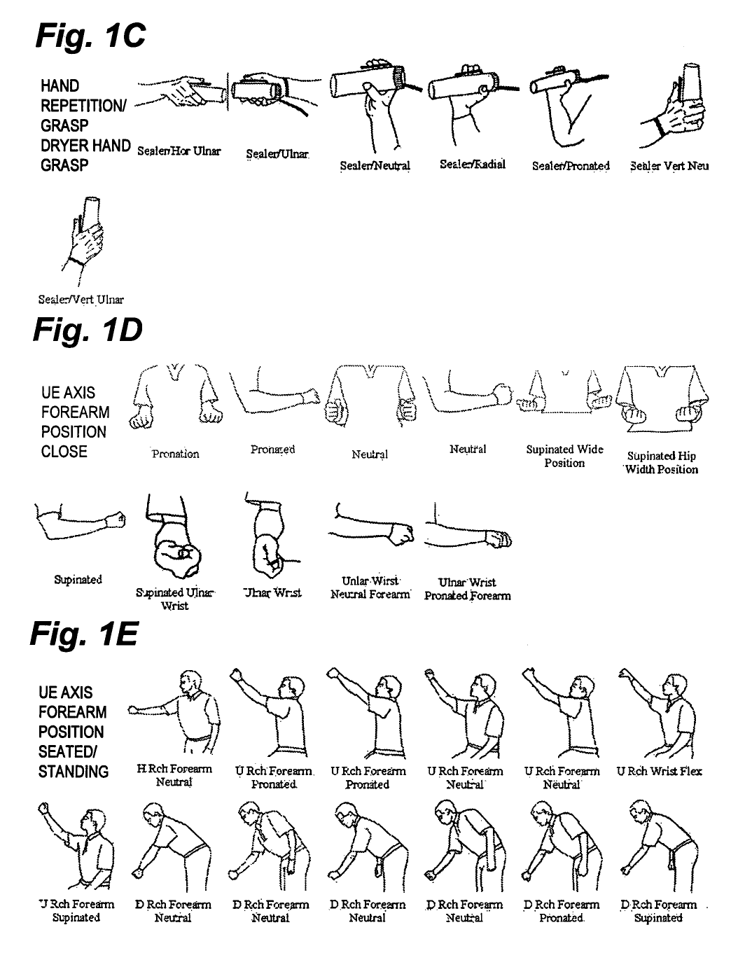

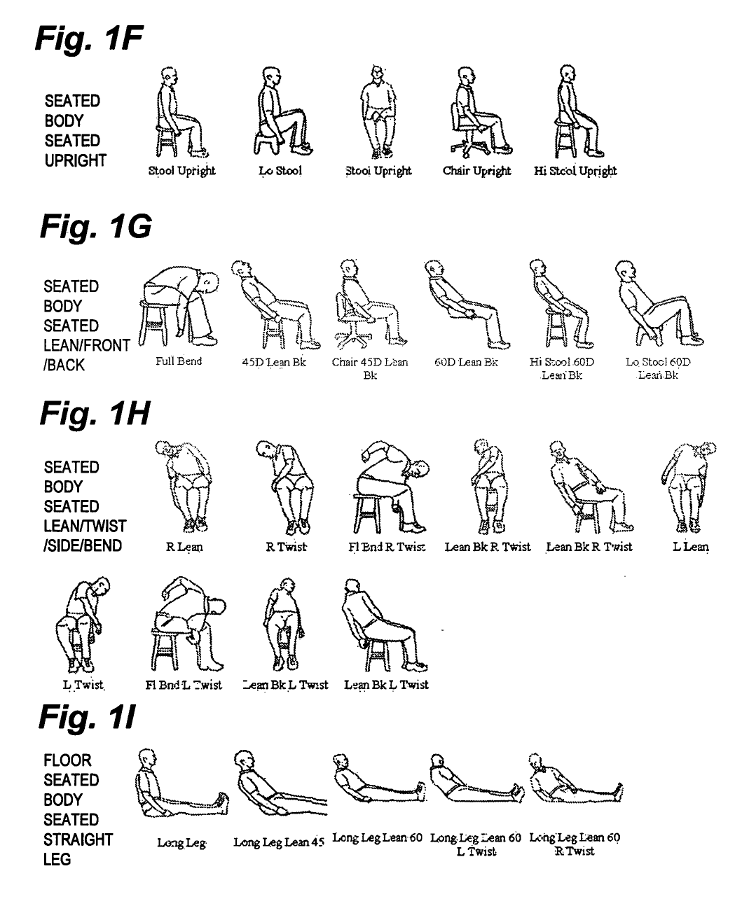

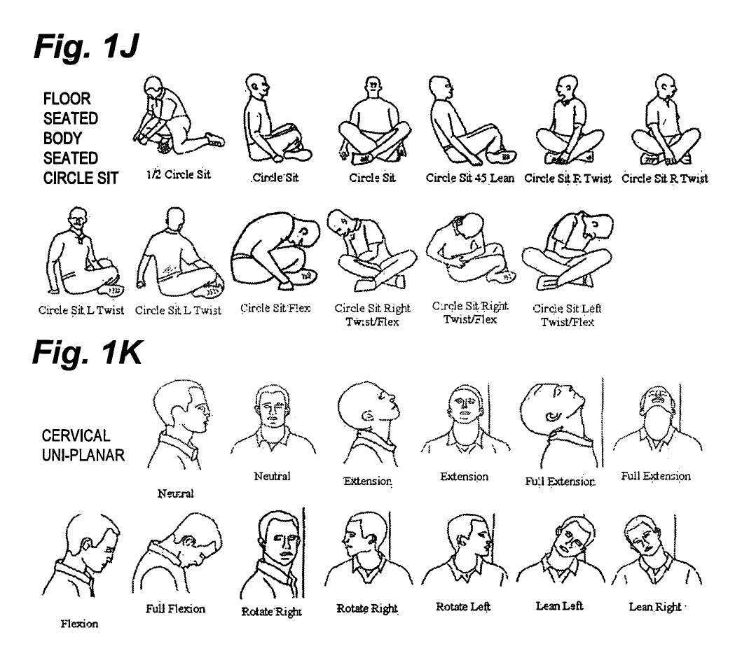

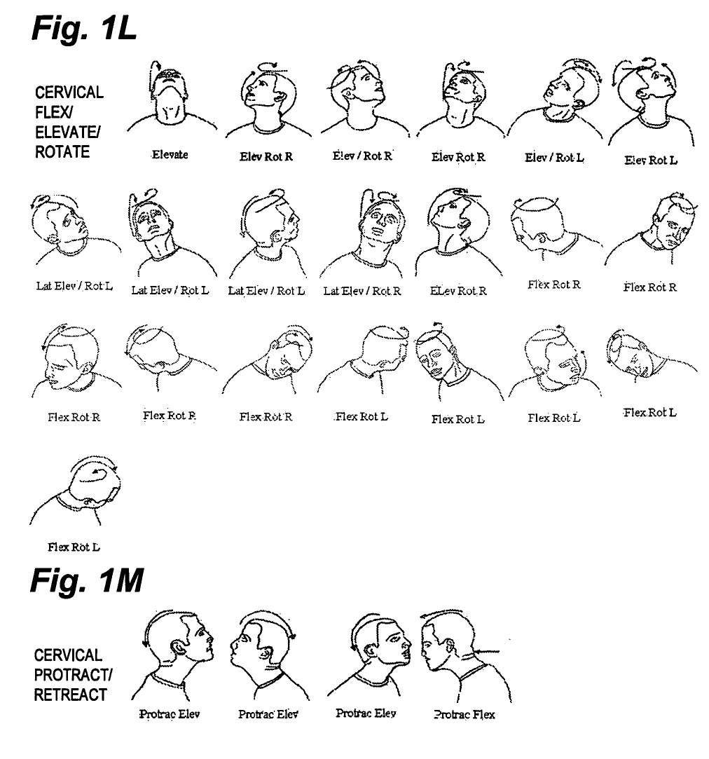

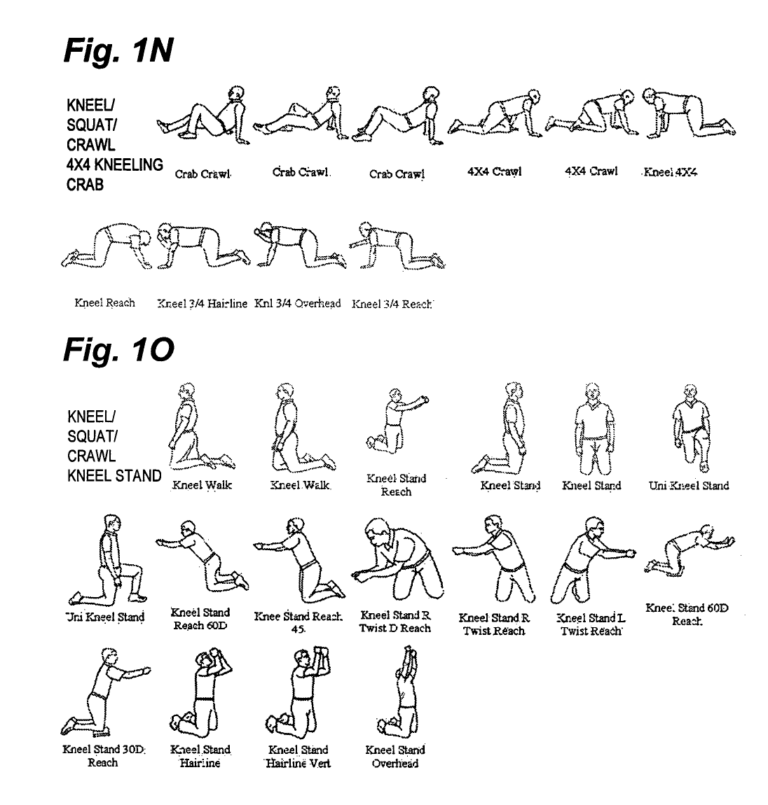

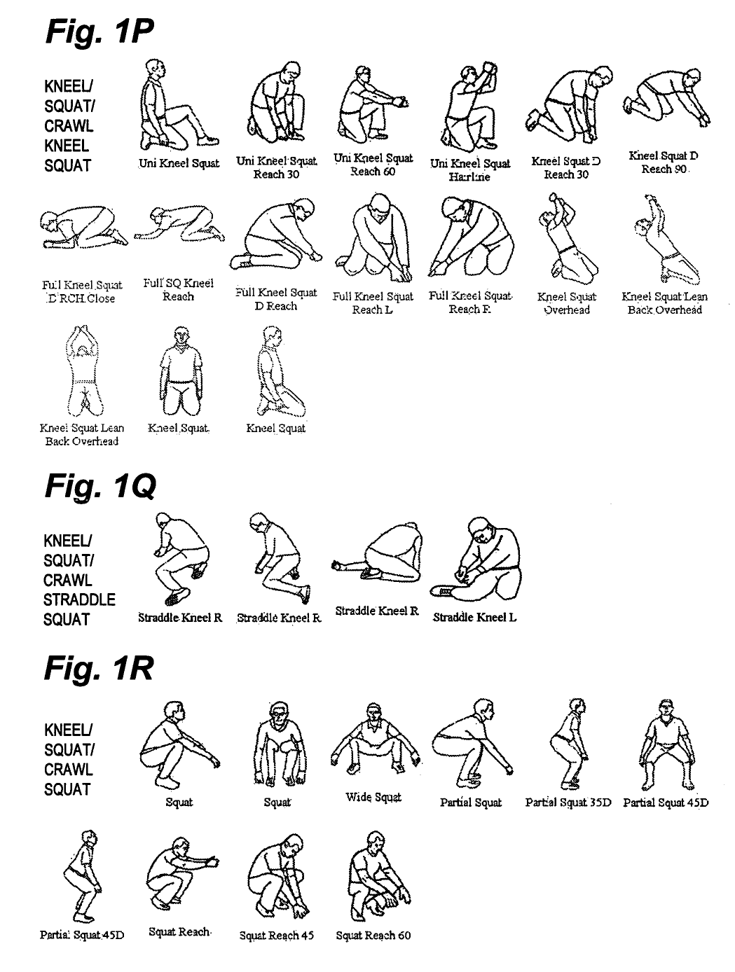

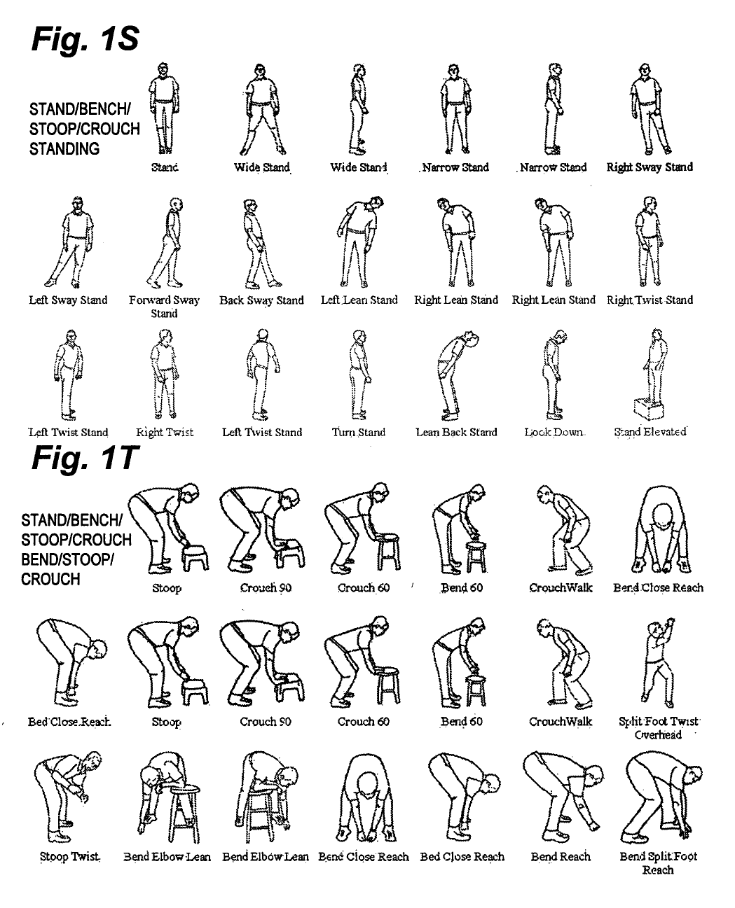

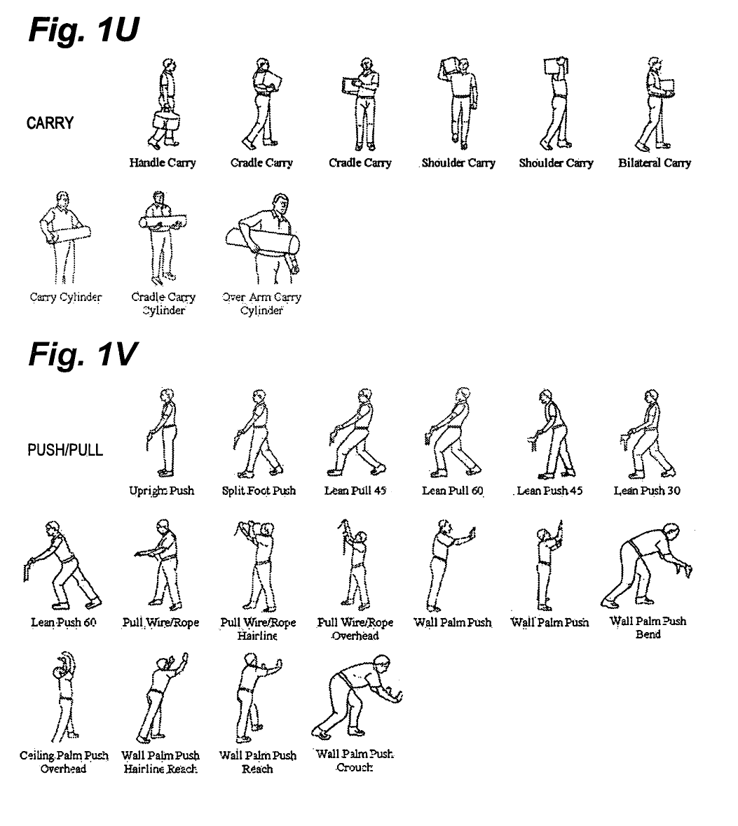

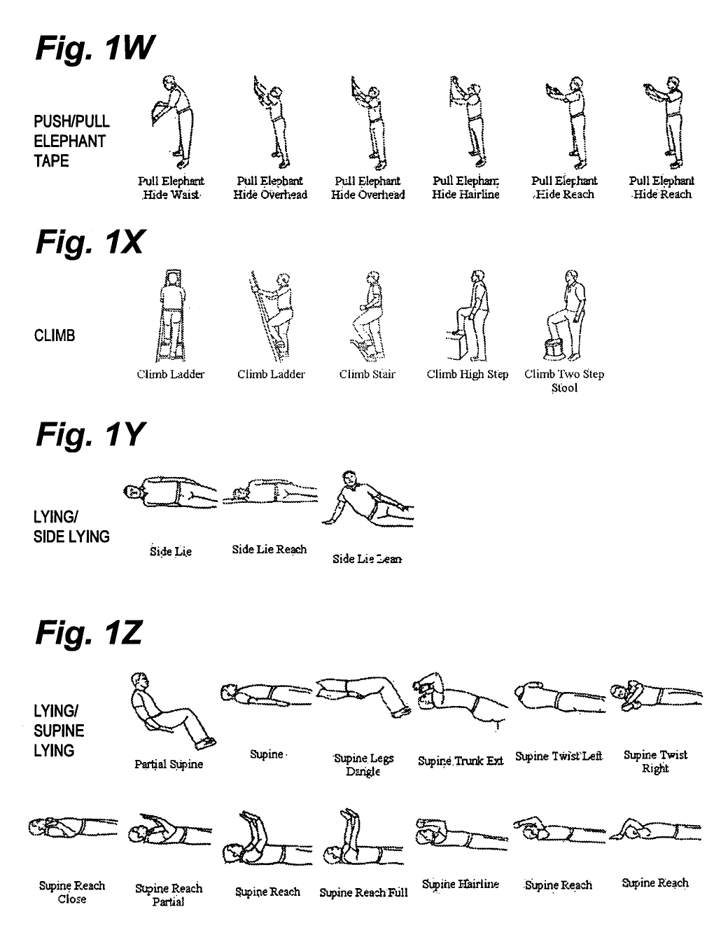

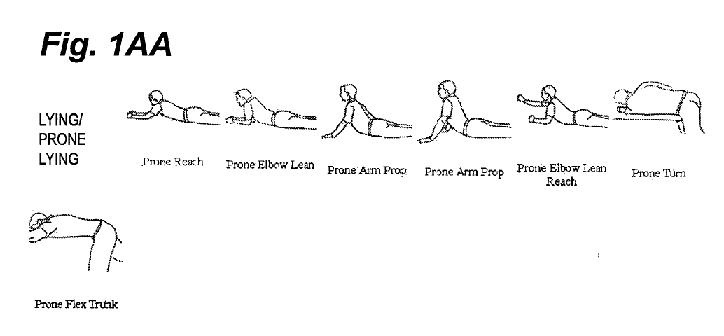

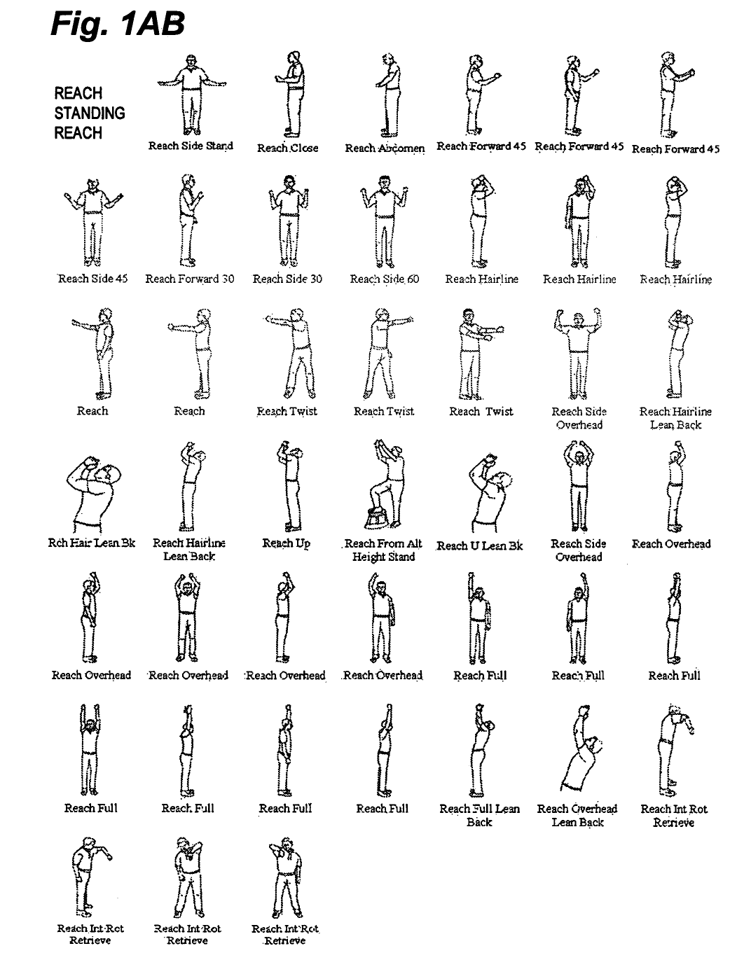

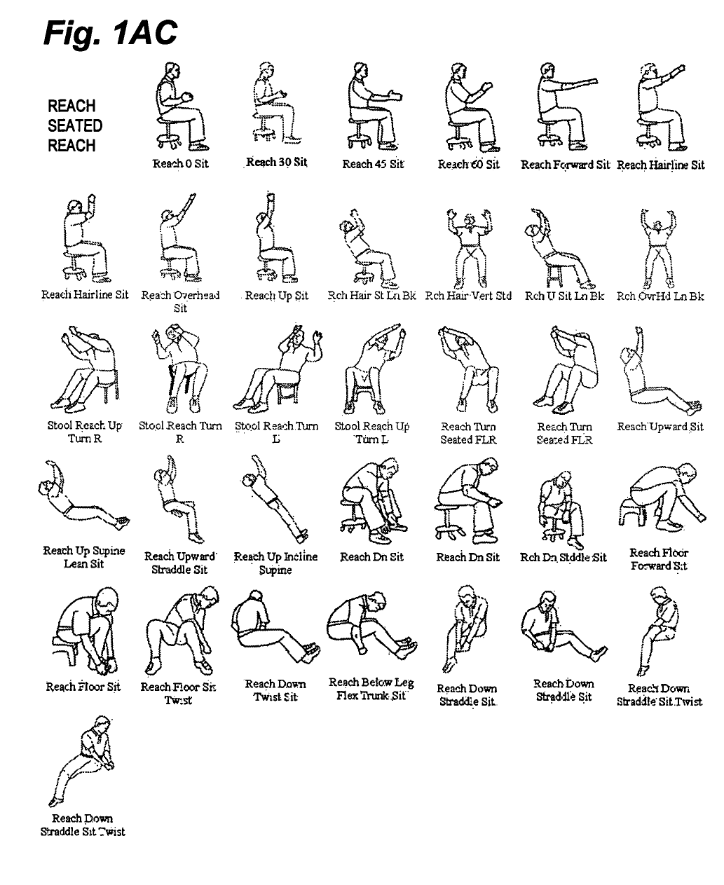

The first step in generating the biomechanical supplement includes selecting biomechanical images from a biomechanical image database stored in a computer readable format for retrieval by a computer. The biomechanical image database represented in FIGS. 1A-1AC includes line art silhouettes representing human body postures or positions and specific body part positions. Each biomechanical image includes a brief text description of the body posture/position or body part position represented in the image. The biomechanical image database can be continually updated with images representing any type of new work posture or position information related to specific industries, work environments or job requirements.

These biomechanical images may be stored in a computer readable database as electronic image files or in a hard-copy printed format. The method of selecting the biomechanical images can easily be accomplished through the use of a graphical user interface device (GUI) on that of a computer, personal digital assistant (PDA), or an interactive communication device such as a cell phone with a capable display. All of these devices have a graphical display and a processor capable of retrieving, arranging, storing and outputting for display or for printing the biomechanical images.

The biomechanical images and FIGS. 1A-1AC are grouped in the electronic database and grouped for display into main categories and subcategories for the purpose of categorizing similar postures and body positions for easier identification purposes. For example, FIGS. 1A-1C are grouped in the main category of “hand repetition/grasp,” with FIG. 1A's subcategory of “fine hand grasp,” FIG. 1B's subcategory of “gross hand grasp,” and FIG. 1C's subcategory of “dryer hand grasp.” FIGS. 1A-1AC depict a universal and comprehensive library or database of biomechanical images not grouped by any specific type of work, but grouped only by similar postures and body positions.

A person selecting biomechanical images from such a universal and comprehensive library or database would need to identify specific biomechanical images associated with a particular job or work function.

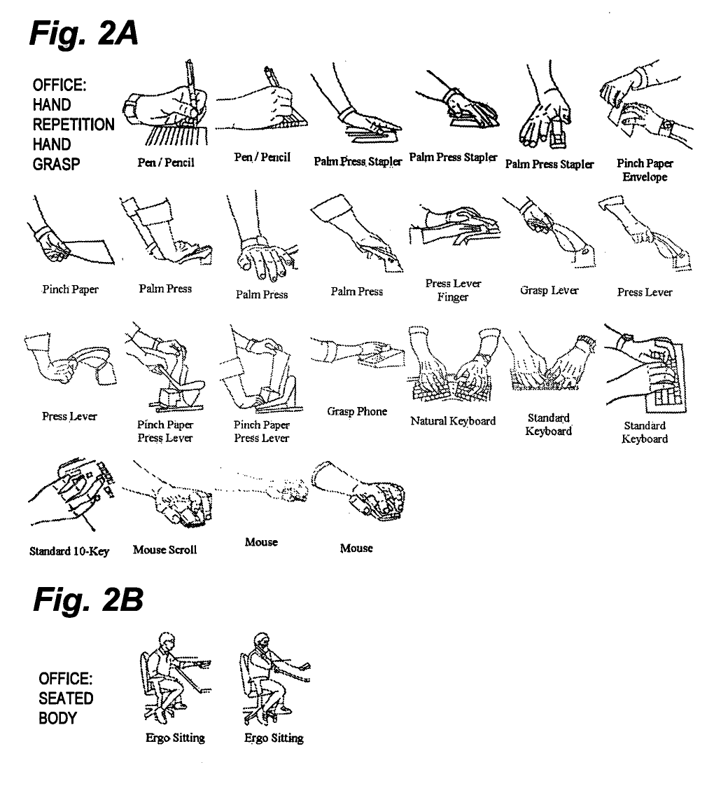

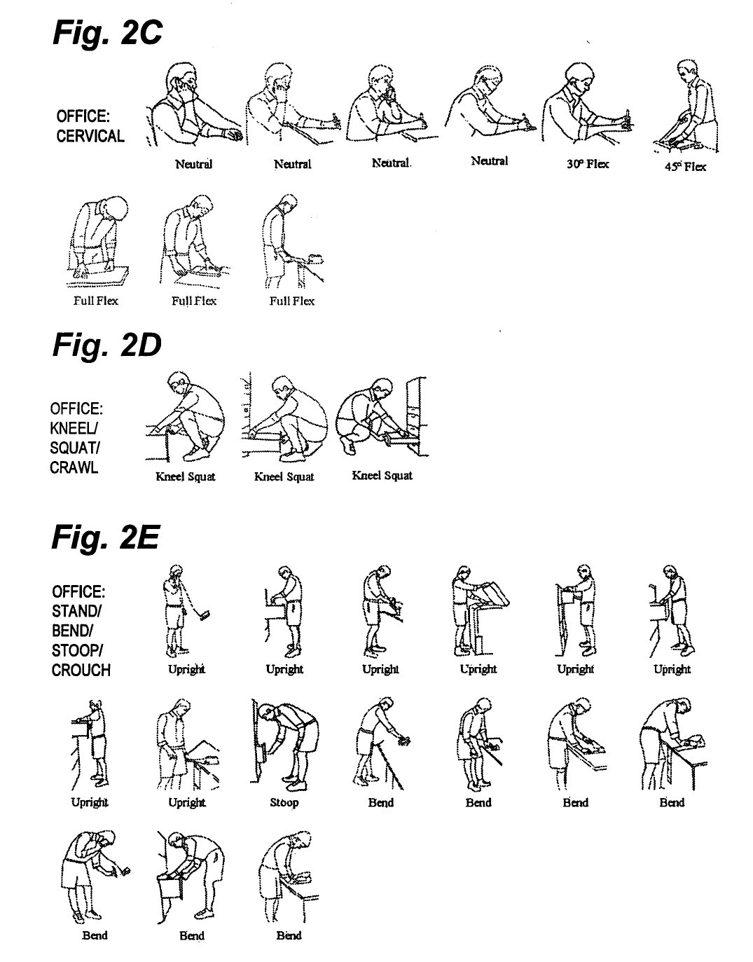

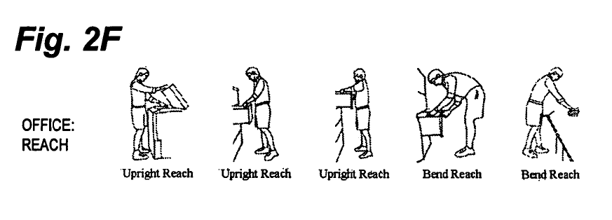

For ease of selecting biomechanical images, an alternative to searching a universal database of biomechanical images would be to search a pre-grouped database of biomechanical images organized by a specific job or work environment. Such is the example of FIGS. 2A-2F wherein biomechanical images are grouped into a representative category of “office” work and are then subcategorized into similar body postures or positions and body positions related to tasks associated with the office environment.

The pre-grouped biomechanical images taken from the universal biomechanical image database may be grouped by any relevant criterion. For example, biomechanical images may be pre-compiled prior to selection based on all varieties of a single titled (main category or subcategory) body posture or position or body part position, body postures or positions or body part positions associated with a particular type of job or work environment, the particular stature of a worker in any particular environment, for a particular type of equipment used on a job or a company's policy with respect to a particular type of equipment used on a job, or body postures or positions or body part positions associated with seasonal changes affecting a job.

After the biomechanical images have been selected from either the universal database or a pre-compiled sub-grouping of biomechanical images, a supplemental description may be assigned to each selected biomechanical image. The supplemental description may include information regarding the frequency of operation associated with the biomechanical image, for example how often the posture or position is used on a specific job or work environment. Additionally, a time period, a load or weight, or a minimum requirement or special requirement associated with the body posture or position or position of the biomechanical image may be assigned as the supplemental description.

The assignment of the supplemental description for a particular biomechanical image may be accomplished in two ways. The first being a free form field wherein a user may input a supplemental description of their own choosing to be associated with the biomechanical image. This may be accomplished by typing via a computer or other text input-enabled device or manually writing the supplemental description with the biomechanical image in a printed format.

The second method for assigning a supplemental description may be the selection of one or more supplemental descriptions from a predetermined list of items. If the assigning of a supplemental description is taking place on an electronic device with a graphical user interface (GUI), a selectable menu may offer the user a list of supplemental descriptions to choose from to associate with a particular biomechanical image. Alternatively, a user may manually choose from a list of supplemental descriptions by identifying their selection of a choice of supplemental descriptions on a printed form that contains the biomechanical image. Supplement descriptions in this alternative may be symbols associated with a common legend capable of being identified and selected by the user.

The final step in assembling the biomechanical images onto the biomechanical supplement document includes organizing or grouping the biomechanical images into similar body posture or position or body part position categories within the document itself. This grouping and identification allows for clear and rapid communication and understanding of the job function capacities based on the grouped biomechanical images.

The completed biomechanical supplement document may then take the form of a printed document, a printed placard or sign, an electronic document including the biomechanical images and supplemental descriptions, an electronic image of a computer-generated or printed documents, or a database record having searchable image and text fields of data.

The printed form of the biomechanical supplement may be printed by a computer controlled printing device to paper media, rigid or board-type media, transparent projection media, or any media suitable for printed publication or graphical communication. The computer controlled printing device may include a computer where a user selects biomechanical images from an electronic image database, the computer then arranges the selected images by type of body posture/position and body part position, and allows the user to input supplemental descriptions to each of the images. Additionally, the computer controlled printing device may include a printer for printing the biomechanical supplement including the selected and organized images and their corresponding supplemental description information.

Likewise, a computer readable medium may store the information of the fully compiled biomechanical supplement and may include an electronic document, such as a word processing file or an cross-platform electronic document (such as an Adobe(R) Portable Document File—PDF), an electronic image of a computer generated or a printed document, such as any type of graphic image file (TIFF, bitmap, JPEG, etc.), a database record capable of storing graphic image data or linking to graphic image database records, or any computer readable medium suitable for storing and presenting a graphical image.

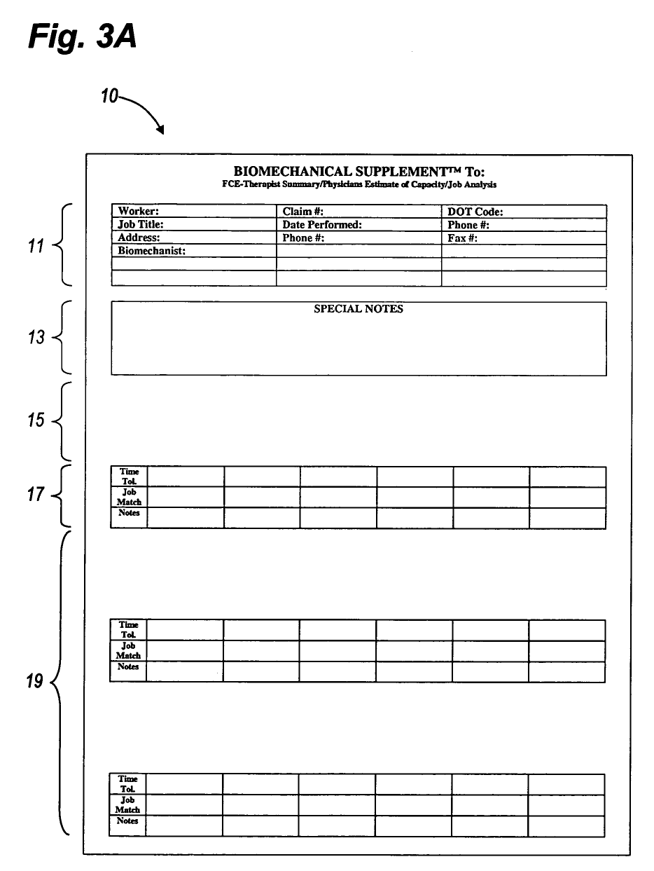

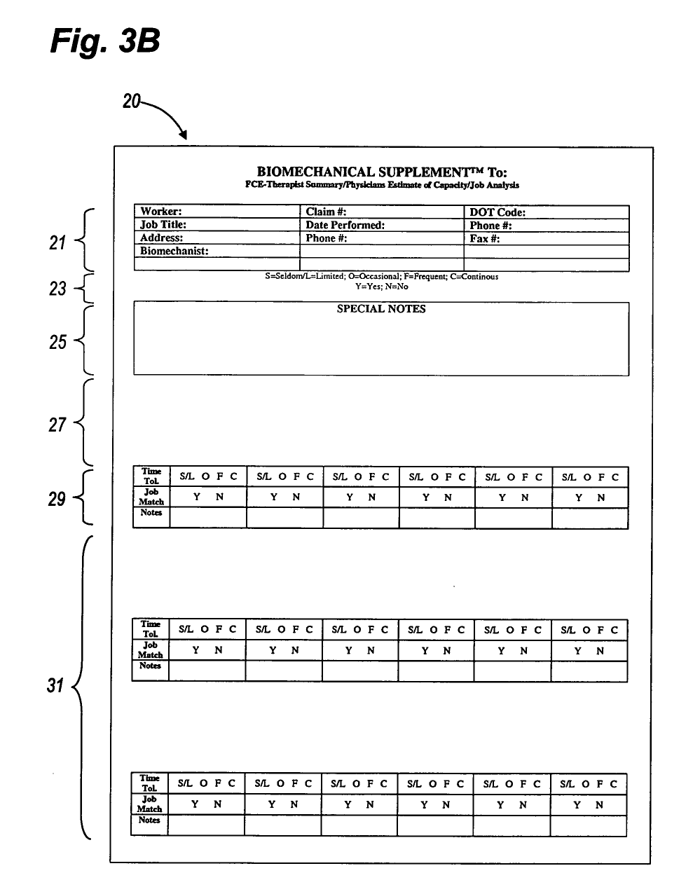

FIG. 3A illustrates a blank biomechanical supplement form 10. A header section 11 includes data fields for identifying a worker, claim number and other relevant case specific information. A special note section 13 includes space for the writer to input additional information as necessary. Reference number 15 indicates the area where the biomechanical images will be placed on the form 10. Below that area is a table 17 for the supplemental description and further notes. For example, the first field in the table for each biomechanical image designates “Time Tol.” for time tolerance to indicate the period of frequency associated with the specific biomechanical image. Underneath, a “Job Match” field may be used to designate whether or not an employee is matched for the function indicated in the associated biomechanical image. These table field designations are for illustration purposes only and are not to be taken as limiting the scope of the present invention to only these types of data. As described above, supplemental description data may be any data that the user deems important and relevant to the biomechanical image associated with it. Reference number 1 9 illustrates the capacity of the biomechanical supplement to include multiple sections of biomechanical images and supplemental descriptions.

FIG. 3B illustrates a biomechanical supplement 20 similar to FIG. 3A. The header section 21, and special notes section 25 remain the same as the previous FIG. 3A. However, a legend 23 illustrates certain characters associated with a frequency of operation, i.e., S=Seldom performed; L=Limited performance; O=Occasionally performed; F=Frequently performed; and, C=Continuously preformed. Reference number 27 designates the area where the biomechanical images are placed. Reference number 29 designates a table where supplemental descriptions can be identified based on characters associated with the legend 23 above. Reference number 31 again illustrates the capacity of the biomechanical supplement to include multiple sections of biomechanical images and supplemental descriptions chosen from a pre-selected list of descriptions.

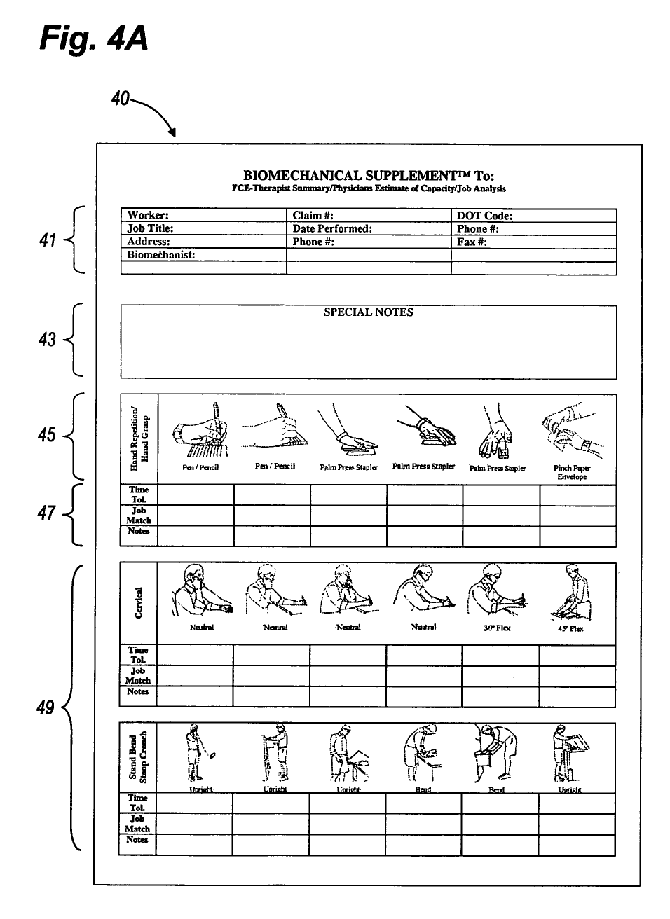

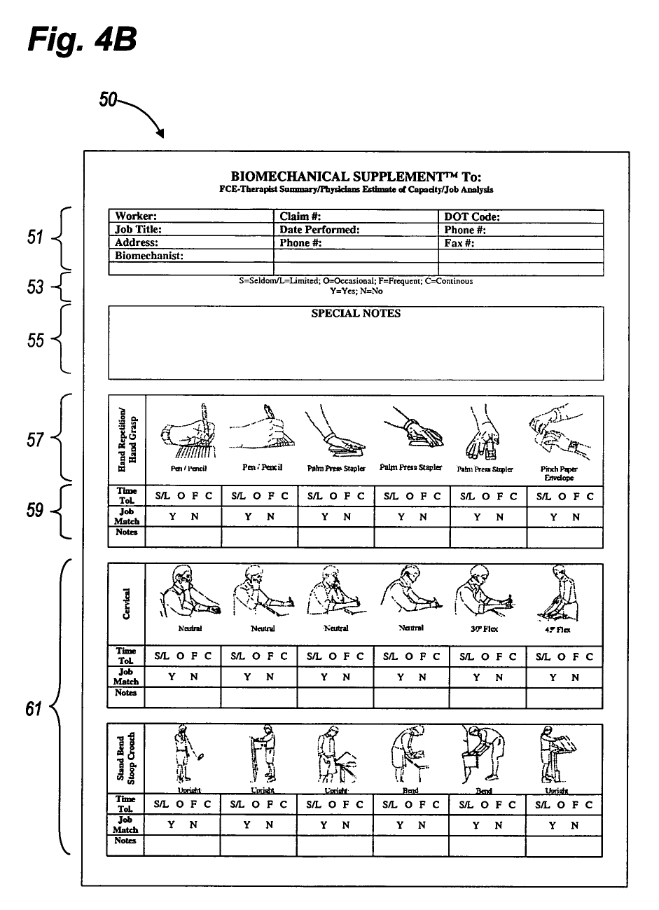

FIG. 4A illustrates a biomechanical supplement 40 including biomechanical images at the section illustrated by reference number 45 but with a blank supplemental description table 47 capable of being filled in by a user. The header information 41 and special note section 43 are identical to the previously identified biomechanical supplements. However, the section represented by reference number 45 now indicates a category description for the biomechanical images on the upper left corner of the supplemental description table. For example, the text “Hand Repetition/Hand Grasp” identifies the following six biomechanical images as being part of that specific biomechanical category. The section represented by reference number 49 illustrates two different distinctive biomechanical categories and their respective biomechanical images.

FIG. 4B illustrates a biomechanical supplement 50 including biomechanical images at 57 now including a supplemental description table 59 associated with a predetermined list of descriptions identified by the legend at 53. Again, the header information 51 and special note section 55 are identical to the previously identified biomechanical supplements. As in FIG. 4A, the section represented by reference number 57 now indicates a category description for the biomechanical images on the upper left corner of the supplemental description table. The section represented by reference number 61 now illustrates two distinctive biomechanical categories, their respective biomechanical images and the predetermined choices for the user to choose their supplemental descriptions.

The disclosed invention of the biomechanical supplement is designed to accurately and quickly convey information to healthcare providers, employers, prospective employees, administrative agencies, triers of fact, etc., regarding a worker's physical capacity, particular job requirements, a forensic tool to identify a person's lack of capacity due to workplace injury, or any other situation where both a quantitative and qualitative description of physical capacity or incapacity is needed.

It is to be understood that the present invention is not limited to the embodiments described above, but encompasses any and all embodiments within the scope of the following claims.

I claim:

1. A computerized method of assembling a plurality of biomechanical images onto a single document, wherein the biomechanical images comprise line graphic silhouettes representing body postures or positions and specific body part positions, the method comprising the steps of:
selecting at least two biomechanical images from an electronic database containing the plurality of biomechanical images;
assigning a corresponding supplemental description to each of said selected biomechanical images; and,
compiling said selected biomechanical images and the corresponding assigned supplemental descriptions onto a single electronic document.

2. The computerized method of assembling a plurality of biomechanical images onto a single document of claim 1, wherein each of the plurality of biomechanical images includes a brief text description.

3. The computerized method of assembling a plurality of biomechanical images into a single document of claim 1, wherein each of the plurality of biomechanical images represents biomechanical images of human bodies and human body parts.

4. The computerized method of assembling a plurality of biomechanical images onto a single document of claim 1, wherein said selecting step further includes the step of selecting a biomechanical image from the group consisting of:
(a) a universal biomechanical image database that includes every selectable biomechanical image, and
(b) a subgrouping of work-specific biomechanical images.

5. The computerized method of assembling a plurality of biomechanical images onto a single document of claim 4, wherein said subgrouping of work-specific biomechanical images further comprises pre-selected subgroupings of biomechanical images selected from the group consisting of:
all varieties of a single titled body posture, whole body position, and body part position;
body postures, whole body positions, and body part positions associated with a particular type of job;
body postures, whole body positions, and body part positions appropriate for the stature of a worker;
body postures, whole body positions, and body part positions associated with a particular type of equipment used on a job;
body postures, whole body positions, and body part positions associated with a company's policy with a particular type of equipment used on a job; and
body postures, whole body positions, and body part positions associated with seasonal changes affecting a job.

6. The computerized method of assembling a plurality of biomechanical images onto a single document of claim 1, wherein said assigning step further,includes an assigning step selected from the group consisting of:
inputting said corresponding supplemental description to be associated with the selected biomechanical image; and
choosing said corresponding supplemental description from a predetermined list of supplemental descriptions to be associated with the selected biomechanical image.

7. The computerized method of assembling a plurality of biomechanical images onto a single document of claim 1, wherein said corresponding supplemental description is selected from the group consisting of:
a frequency of operation description associated with a body posture, whole body position and body part position;
a time period description associated with a body posture, whole body position, and body part position;
a load associated with a body postures, whole body positions, and body part position; and
a minimum or special requirements description associated with a body postures, whole body positions, and body part position.

8. The computerized method of assembling a plurality of biomechanical images onto a single document of claim 1, wherein said compiling step further comprises the step of organizing and grouping the selected biomechanical images into similar body postures, whole body positions, and body part position categories on said single document.

9. The computerized method of assembling a plurality of biomechanical images onto a single document of claim 1, further comprising the step of printing said electronic document.

10. The computerized method of assembling a plurality of biomechanical images onto a single document of claim 1, wherein the single document comprises a job analysis.

11. The computerized method of assembling a plurality of biomechanical images onto a single document of claim 1, wherein the single document comprises a biomechanical supplement to a job analysis.

12. A computerized system for inserting user-selectable biomechanical graphic images into a job analysis, comprising:
a computerized device having a microprocessor, an area of main memory, a storage memory, a graphical use interface, and a data bus connecting the microprocessor, the main memory, the storage memory, and the graphical user interface;
a database having stored therein a plurality of graphical biomechanical images forming a comprehensive biomechanical database of body postures, whole body positions, and body part positions relating to functions performed in a plurality of occupations;
software means for preparing the job analysis stored in the storage memory and executable in the main memory under control of the microprocessor, the means for preparing the job analysis including:
means for retrieving the biomechanical images from the database and viewing the images on the graphical user interface; and
means for allowing the user to select and insert at least one of the viewed biomechanical images into a textual job analysis document in order to illustrate the biomechanical skills involved in performing a particular job.

13. The computerized system according to claim 12, wherein said computerized device is a standalone workstation, said database being stored in the storage memory of the workstation.

14. The computerized system according to claim 12, wherein said computerized device is a client workstation having means for accessing a network, said database being stored on a server computer accessible to the client workstation through the network.

15. The computerized system according to claim 12, wherein said means for allowing the user to select and insert at least one of the viewed biomechanical images comprises means for allowing the user to select and insert a plurality of the viewed biomechanical images into the textual job analysis document.

16. The computerized system according to claim 12, further comprising means for printing the job analysis document with the at least one graphical biomechanical image incorporated therein.

17. A computer software product that includes a medium readable by a processor, the medium having stored thereon a set of instructions for preparing a job analysis having graphical biomechanical images incorporated therein, comprising:
(a) a first sequence of instructions which, when executed by the processor, causes said processor to retrieve the biomechanical images from a comprehensive database of graphical biomechanical images;
(b) a second sequence of instructions which, when executed by the processor, causes said processor to display the images for viewing on a graphical user interface;
(c) a third sequence of instructions which, when executed by the processor, causes said processor to insert at least one of the viewed biomechanical images selected by a user into a textual job analysis document in order to illustrate the biomechanical skills involved in performing a particular job.

18. The computer software product according to claim 17, wherein said computer readable medium has said database stored thereon.
